(19) United States
(12) Patent Application Publication
(10) Pub. No.: US 2009/0281568 A1
(43) Pub. Date: Nov. 12, 2009

(54) Devices and Methods for Adjustable, Knotless Tissue Approximation

(76) Inventors: Juan Carlos Cendan, Gainesville, FL (US); Richard Helmig, Hawthorne, FL (US)

Correspondence Address: SALIWANCHIK LLOYD & SALIWANCHIK;A PROFESSIONAL ASSOCIATION, PO Box 142950, GAINESVILLE, FL, 32614, US

(21) Appl. No.: 12/303,809

(22) PCT Filed: Jun. 14, 2007
(86) PCT No.: PCT/US07/71220

§ 371 Date: Jul. 13, 2009

Related U.S. Application Data
(60) Provisional application No. 60/813,398, filed on Jun. 14, 2006.

(51) Int. Cl. A61B 17/08

(52) U.S. Cl. 606/217

(57) ABSTRACT

Devices and methods for tissue approximation and distribution of tissue stresses and tension resulting therefrom. The devices comprise a shank having one or more structures thereon, an anchor button fixedly attached to one end of the shank with the opposite end of the shank being suitable for penetration of tissues or other structures. To secure the device, the shank engages with a locking button having a locking mechanism thereon. The locking button is engaged with the shank, preferably after a tissue approximation, so that the locking button can be advanced along the shank towards the anchor button compressing the approximated tissues between the anchor button and the locking button.

### BACKGROUND OF INVENTION

Tissue approximation is a necessary step in many surgical procedures. It can be a particularly difficult step when tissue has been excised or otherwise displaced from a wound area leaving a gap in the tissues. In such situations, surrounding tissues must often be repositioned by pulling or stretching to cover or close-over the area of the excised or missing tissues.

Current surgical techniques often utilize surgical sutures to pull or stretch surrounding tissues to accomplish tissue approximation. For example, hiatal hernia repair often requires that an enlarged opening occurring in the wall of the diaphragm be closed by stretching the surrounding diaphragmatic tissues over the opening. Commonly used sutures for these techniques comprise various gage threads often made of silk, various nylons, monofilaments, wires, or various bioabsorbable materials and/or combinations thereof. Using sutures of this type requires that the ends of the threads be knotted to properly approximate and hold the tissues.

For external wounds or “open cavity” surgical procedures, the use of standard sutures and knot tying is usually not problematic. However, intracorporeal suturing is generally considered challenging for some surgeons, and more so when required with high-tension tissue approximations. For example, in areas of large tissue excision, the surrounding tissues often must be pulled or stretched over a greater area causing considerable tension across the tissue. The use of sutures in these situations can lead to further damage. It is generally thought that pressures exceeding the mean capillary filling pressure of 25-30 mmHg will lead to tissue necroses. Further damage can occur when the approximated tissue area is pulled out of the sutures, which cuts or shreds the edges of the formerly approximated tissues. The use of meshes and certain mechanical tissue supports have been described in the art to buttress such closures, but they are not always effective and complications have been associated with these techniques and materials.

In addition, it is also more difficult in certain types of surgeries to tie the various specialized knots required with the use of sutures. For example, various endoscopic surgeries, e.g., laparoscopic, rhinoscopic, colonoscopic, often require the use of diminutive instruments, with little or no tactile feedback, in confined spaces. In these situations, knot-tying could be impossible, or at least more difficult and time-consuming.

Several alternatives to sutures have been devised that can approximate tissues without sutures or the need to tie knots. For example, U.S. Pat. No. 6,074,401 and U.S. Published Applications 2004/0044364 and 2005/0228415 describe devices and methods for approximating or joining tissues that do not require the use of sutures or knots and can be used in endoscopic surgeries. However, while these devices are designed to join or hold tissues together, they may not be particularly effective in techniques where tissues are stretched or pulled and then held securely to close an opening. These devices also do not provide sufficient means to adjust the pressure applied to the approximated tissues. And, further, these devices require a specialized installation tool.

Other devices can be used to hold tissues, particularly bone tissue, in a certain position for proper healing. For example, U.S. Pat. Nos. 6,022,351 (Bremer) and 6,485,493 (Bremer et al.) describe closure devices for skull flaps that properly position and hold a skull bone fragment within a skull opening for proper alignment and healing. However, these device require that an opening exist through which the shank portions can be positioned so that the disc shaped ends can be pressed together.

The subject invention provides materials and methods for soft tissue approximation (and other surgical applications, such as trocar site closure). The practice of the subject invention does not require specialized tools for installation, which makes it particularly suited for endoscopic surgeries where space can be at a minimum. In addition, the invention allows for greater control over the amount of pressure applied to approximated tissues, without the need to tie knots. Further, the devices of the subject invention are able to distribute tension, which often occurs in approximated tissues, over a broader area so as to reduce or prevent tearing and/or failure of the tissue approximation.

### BRIEF SUMMARY

The subject invention provides materials and methods for tissue approximation and similar tissue closing procedures. Advantageously, the materials and methods of the subject invention can be used with existing surgical tools and are well-suited for endoscopic surgeries. As described herein, devices of the subject invention are particularly useful for modest tissue-tension repairs, for example, hernia repairs and for tissue closure procedures such as, for example, trocar site closure. These devices eliminate (or at least reduce) the need for sutures and the associated knot-tying, and allow for accurate control of the pressure applied to approximated tissues. These devices can also reduce or eliminate failure of tissue approximations and the associated need for additional surgeries to re-approximate damaged tissues.

In one embodiment, the devices of the subject invention comprise a shank with a relatively broad button at one end, wherein the shank has one or more ratchet teeth, beads, fins, or other notched projections on at least one side, and a needle, or other sharp end implement, fixedly attached or otherwise joined to the end opposite the button. The needle is used to penetrate tissues to be approximated and held together. Once the tissues or other materials have been threaded onto the length of the shank, the needle is passed through an opening in a second relatively broad locking button. The locking button mechanism can be, for example, one or more pawls that correspond to the one or more ratchet teeth on the shank, such that the locking button can be pushed, or otherwise moved, along the length of the shank so that the ratchet teeth and pawl engage to prevent, or at least resist, the locking button from moving backwards on the shank. In this way, the two buttons can be pushed together to approximate the tissues therebetween.

Alternatively, the devices of the subject invention can be used to seal or plug an opening in tissues that cannot, or should not, be approximated, such as, for example, trocar sites. In this procedure, the buttons of the device can be placed on either side of a tissue opening, such that the edges of the buttons overlap the edges of the tissue opening, and can be drawn together utilizing the shank between them until they are against either side of opening.

The width of the shank and the shape and diameter of the buttons help to distribute tension, caused by pulling or stretching the tissues, over a greater area. This reduces the pressure on the tissues around the shank and reduces or eliminates tearing or shredding of the approximated tissues. This can help to prevent failure of the tissue approximation and the need for additional surgery to re-approximate tissues.

### BRIEF DESCRIPTION OF DRAWINGS

FIG. 1A is an isometric view of an embodiment of a tissue approximation device of the subject invention.

FIG. 1B is an isometric view showing alternative embodiments of various components of a tissue approximation device of the subject invention.

FIG. 1C is an isometric view showing an alternative embodiment of the shank that can be utilized with the devices of the subject invention. This embodiment utilizes a generally oval cross-sectional shaped shank, with ratchet teeth on at least one side.

FIG. 1D is an isometric view showing an alternative embodiment of the shank that can be utilized with the devices of the subject invention. This embodiment utilizes a generally rectangular cross-sectional shaped shank, with ratchet teeth on two or more sides.

FIGS. 1E-1G are photographs of one embodiment of the devices of the subject invention.

FIG. 1H is an illustration of a device of the subject invention utilizing a shank comprising a suture having a needle attached or otherwise joined to the distal end for tissue penetration.

FIG. 2A is a cross-sectional view of two columns of tissue that have been approximated with an embodiment of the tissue approximation device of the subject invention.

FIG. 2B is a photograph of tissue approximated with standard sutures and surgical techniques. Note the constriction of the tissues around the sutures.

FIG. 3A is an illustration of one alternative embodiment of the device of the subject invention. This embodiment utilizes anchor and locking buttons having projections thereon for penetrating tissues to stabilize the device and/or surrounding tissues.

FIG. 3B is an illustration of another alternative embodiment of the device of the subject invention. This embodiment, utilizes an anchor button modified with hooks for penetrating tissues to stabilize the device and/or surrounding tissues.

FIG. 4A is an illustration of a still further alternative embodiment of the device of the subject invention. This embodiment utilizes an anchor button modified as a toggle-end device for penetrating tissues or other materials.

FIG. 4B is an illustration of yet a further alternative embodiment of the device of the subject invention. This embodiment utilizes an anchor button modified as a harpoon-like device for penetrating tissues or other materials.

FIG. 4C is an illustration of how the embodiments shown in FIGS. 4A and 4B can be utilized to stabilize or support tissues with a surgical mesh apparatus.

FIG. 5A is an illustration of a still further alternative embodiment of the device of the subject invention. This embodiment utilizes a shank modified with a plurality of fins or rib-like structures for securing the shank with the locking button.

FIG. 5B is a cross-sectional view taken along long A-A′ of the device in FIG. 5A.

FIG. 5C is a front plan view of an alternative embodiment of a locking button. This embodiment utilizes a semi-rigid material for the locking button, or at least around the periphery of the shank slot. The semi-rigid material can be bent or otherwise temporarily deformed to permit the passage of a shank. This embodiment is particularly useful with firmed and/or beaded shanks.

FIG. 5D is a side plan view of the alternative embodiment shown in FIG. 5C. FIG. 6A is an illustration of a still further alternative embodiment of the device of the subject invention. This embodiment utilizes a shank modified with a plurality of beads or similar structures for securing the shank with the locking button.

FIG. 6B is a cross-sectional view taken along long B-B′ of the device in FIG. 6A.

FIG. 6C is a front plan view of the pressure face of an alternative embodiment of a locking button. This embodiment utilizes one or more shoulder flanges to restrict the size of the shank slot. The shoulder flanges can comprise a semi-rigid material that can be bent or otherwise temporarily deformed to permit the passage of a shank. This embodiment is particularly useful with finned and/or beaded shanks. FIG. 6D is a side plan view of the alternative embodiment shown in FIG. 5C.

FIG. 7A is an illustration of a still further alternative embodiment of a locking button of the subject invention. This embodiment utilizes one or more shank slot tabs in conjunction with one or more shoulder flanges.

FIG. 7B is a side plan view of the alternative embodiment shown in FIG. 7A.

FIG. 7C is an illustration of the locking button shown in FIGS. 7A and 7B being utilized with a finned shank.

### DETAILED DISCLOSURE

The subject invention provides devices and methods for tissue approximation or for sealing or plugging openings in tissues that cannot, or should not, be approximated. Advantageously, the tissue approximation devices of the subject invention can be used with existing surgical tools and are well-suited for endoscopic surgeries. As described herein, the devices of the subject invention are particularly useful for modest tissue-tension repairs. Specifically, exemplified herein is the use of the novel tissue approximation devices of the subject invention for hernia repairs.

The devices of the subject invention eliminate (or at least reduce) the need for sutures and the associated knot-tying, and allow for accurate control of the pressure applied to approximated tissues. These devices can also reduce or eliminate failure of tissue approximations and the associated need for additional surgeries to re-approximate damaged tissues.

In a specific embodiment, a device of the subject invention can comprise a shank with a relatively broad button at one end (the proximal end), wherein the shank has a plurality of ratchet teeth on at least one side and a needle or other tissue penetrating device fixedly attached or otherwise joined to the end opposite the button (the distal end). The needle is used to penetrate tissues, tissue support apparatuses, or other materials, e.g., surgical mesh, prosthetics, or similar medical apparatuses, to be approximated and held together. Once the tissues have been threaded onto the length of the shank, the needle is passed through an opening in a second relatively broad locking button. The locking button mechanism can be, for example, one or more pawls that correspond to the plurality of ratchet teeth on the shank, such that the locking button can be pushed, or otherwise moved, along the length of the shank so that the ratchet teeth and pawl prevent, or at least resist, the locking button from moving backwards on the shank. In this way, the two buttons can be pushed together to approximate the tissues therebetween.

The width of the shank and the shape and diameter (or circumference) of the buttons help to distribute tension caused by pulling or stretching of the tissues, over a greater area. This reduces the pressure on the tissues around the shank and reduces or eliminates tearing or shredding of the approximated tissues. This can help to prevent failure of the tissue approximation and the need for additional surgery to re-approximate tissues.

Embodiments of the tissue approximation devices of the subject invention can comprise any of a variety of materials and, if desired, various parts of the devices can comprise the same or different material(s). The material(s) utilized for the devices will depend upon the anticipated applications of the devices, as well as the environmental conditions and tissues to be approximated. For example, it can be preferable that the subject devices when utilized for hiatal hernia repairs comprise permanent, non-absorbable materials. But, devices of the subject invention utilized for trocar site closures or installation of abdominal meshes preferably comprise less permanent, bioabsorbable materials. Therefore, various components of the devices can comprise any of a variety of biocompatible materials, such as silicone, plastic, biocompatible glass, titanium, stainless steel, etc. In alternative embodiments, one or more bioabsorbable material(s) can be utilized, for example, catgut, Vicril, or POLYSORB. In yet further alternative embodiments, more permanent or non-bioabsorbable materials can also be utilized, for example, silk, nylon, stainless steel, polypropylene, or combinations thereof.

In a preferred embodiment, the devices comprise one or more biocompatible materials with sufficient strength to withstand necessary pulling, tension or compression as required to accomplish a tissue approximation and ultimate assembly of the device. To illustrate, a shank or some length thereof, would require sufficient stiffness of form to permit penetration of muscle, tissue or fascia, but be soft and/or resilient enough to be cut with one or more, typical, surgical implements. Any of a variety of materials are known, and a person with skill in the art would be able to determine the appropriate material(s) for the various uses for which embodiments of the tissue fastener devices of the subject invention can be utilized.

In addition, various components of the devices of the subject invention can further be any of a number of sizes and shapes depending upon the anticipated application of the device. For example, certain embodiments of the device can be used for the repair of problematic hiatal hernias. This type of repair usually requires that the diameter of the opening in the diaphragm through which the esophagus normally extends be reduced to prevent the stomach from also protruding into the chest cavity. The diaphragm is the primary muscle of respiration, which makes it very strong and in a constant, cyclic motion. Repair of a hernia in this constantly moving powerful muscle requires that the approximated tissues be held firmly and securely enough to allow healing, but still withstand the cyclic forces exerted across the muscle. Therefore, the devices of the subject invention could comprise larger dimensions to withstand these forces and a relatively strong biocompatible material that will remain stable for several weeks while the repair heals, or be permanent. For example, one embodiment of the subject invention for hiatal hernia repair would comprise buttons, as described below, of a biocompatible material having diameters of approximately 5.0 mm to about 10.0 mm. In a more preferred embodiment, the button diameters would be approximately 7.0 mm to about 9.0 mm in diameter. Conversely, repairs in smaller or more stable tissues can utilize devices of smaller proportions or biocompatible materials with various absorption rates.

It is known that the amount of pressure exerted on tissue, or any other object, is inversely proportional to the area upon which the force is exerted, i.e., pressure=force/area. Thus, a person with skill in the art would be able to determine the appropriate size and materials for the device required to approximate tissues and for use, if necessary, with the appropriate size trocar or similar installation device. For example, Table 1 shows the proportional difference between cross-sectional diameters of sutures known in the art and a device of the subject invention. It can be seen, for example, that a device of the subject invention utilizing a 6 mm button when compared to a #1 suture having a cross-sectional diameter of 0.4 mm has approximately 225 times more proportional surface area for tissue contact.

TABLE 1

| USP Suture | Diameter (cross-sectional), | |
|---|---|---|
| Designation | non-absorbable suture (mm) | Area (mm²) |
| 2-0 | 0.3 | 0.070686 |
| 0 | 0.35 | 0.096211 |
| #1 | 0.4 | 0.125664 |
| 6 mm button | 6.0 | 28.27431 |

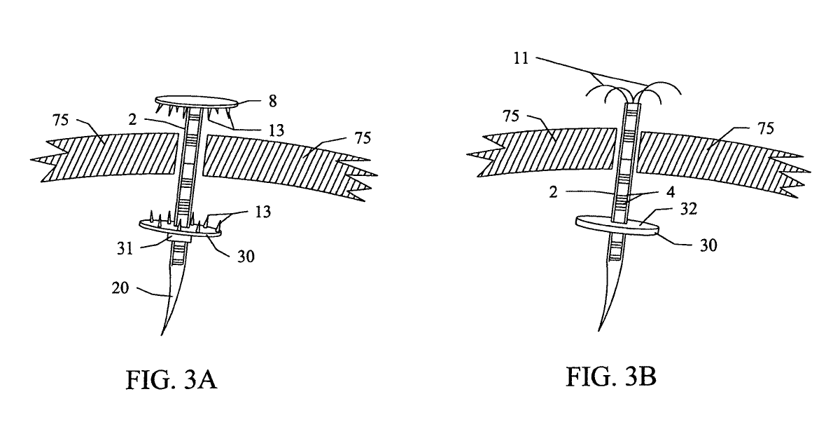

In another example, certain embodiments of the device can be used for the repair of large trocar sites, often necessitated by laparoscopic surgical procedures. This type of repair can utilize the anchor and lock buttons of the subject device as a means for closing the trocar opening without pulling or stretching tissues across the opening. In this embodiment, an example of which is shown in FIGS. 3A and 3B, each button of the device can be placed on either side of a tissue opening, such that some portion of the edges of the buttons overlap the edges of the tissue opening, with the shank extending through the tissue opening. Utilizing the shank, the buttons can be drawn together and into contact with tissue on either side of and around the trocar opening. In this way, the trocar site is closed by the buttons of the device. In a further embodiment, the subject device can comprise one or more bioabsorbable materials that over time will dissolve allowing full closure of the trocar site.

In a still further embodiment, one or both buttons can comprise any of a variety of hooks, knobs, teeth, pressure devices, gripping elements, clamps, adhesives, or other stabilizing devices or fixtures to maintain the placement of the anchor button against tissue. In one embodiment, the face or contact surface of one or both buttons comprises hooks that are bent, angled or otherwise pointed in the general direction of the face of the opposite button. In this embodiment, when the buttons are drawn together, the hooks penetrate or press into tissues, stabilizing the buttons and preventing them from being twisted or turned. A further alternative embodiment can utilize magnetic materials that permit the coupling or “connection” of the buttons either directly or through tissues or structures by magnetic force.

In further embodiments, these devices can also be impregnated with any of a variety of medications or drugs, for example, pain reducers, anti-inflammatories, antibacterials, growth hormones, etc. can be incorporated into or on these devices by techniques known to those with skill in the art. In still further embodiments, the devices could comprise materials that aid in their visualization with, for example, radiographic, MRI, CTI, PET, or other imaging equipment. A person with skill in the art would be able to determine from any of a variety of materials, which would be appropriate for such visualization scans.

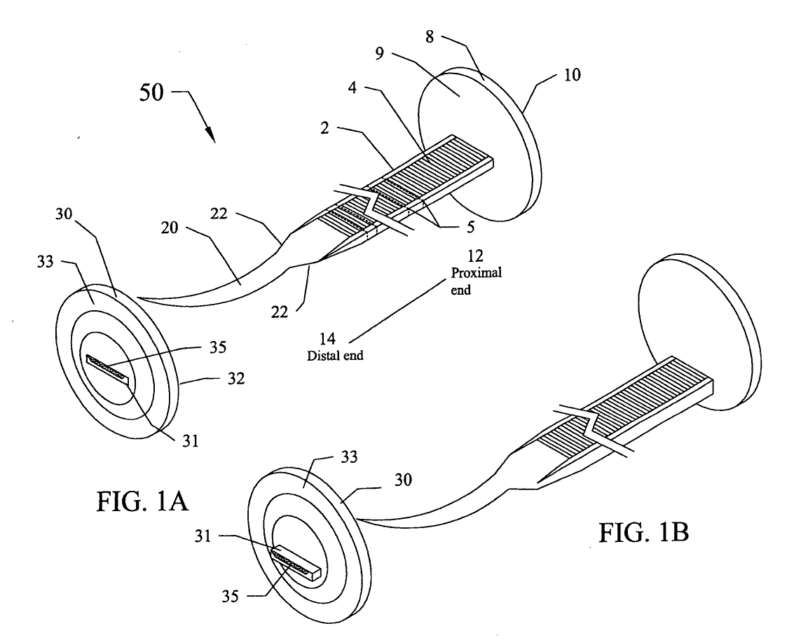

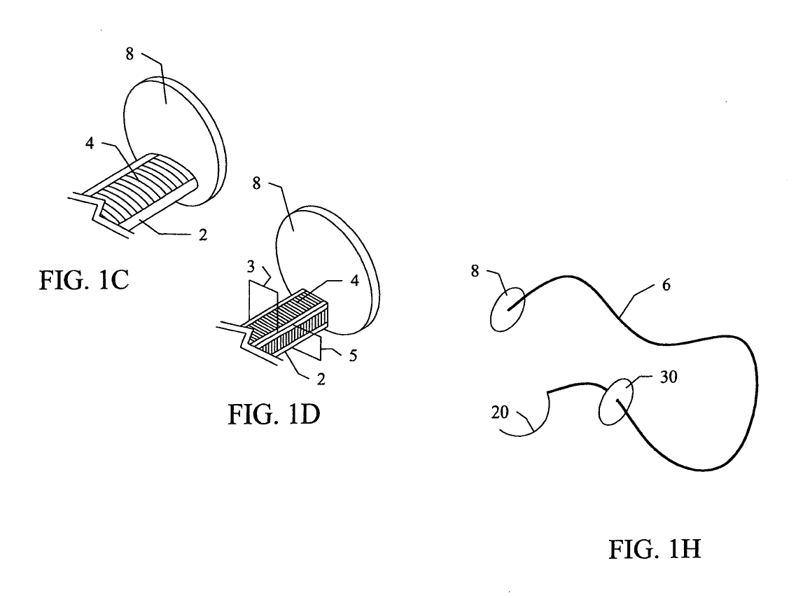

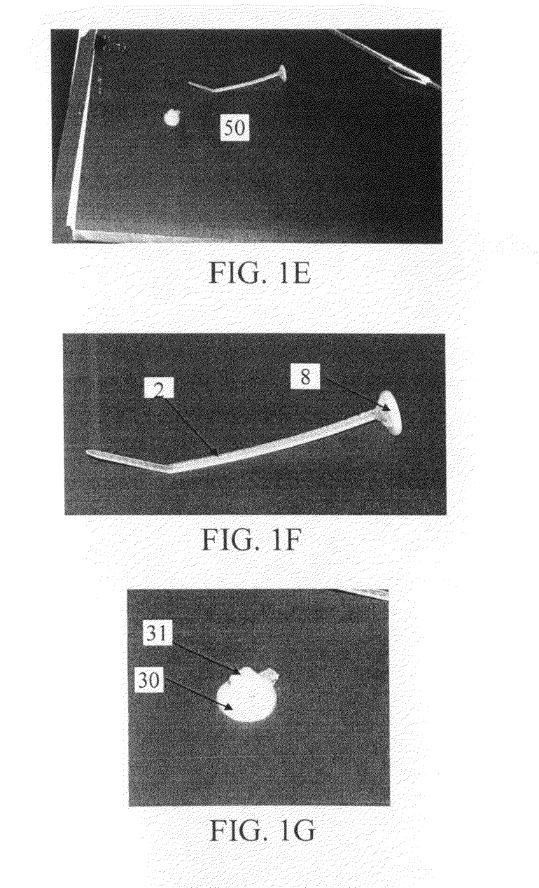

With reference to the attached figures, which show certain embodiments of the devices of the subject invention, it can be seen that the devices 50 of the subject invention comprise at least one elongated shank 2 having a proximal end 12 and a distal end 14 with a plurality of notched projections, such as, for example, beads, fins, and/or ratchet teeth 4 thereon. In one embodiment, shown for example in FIG. 1A, the device 50 comprises a single shank 2, but alternative embodiments can utilize two or more shanks 2. As mentioned previously, the shank 2 can be any desired length or width required to accomplish the desired tissue approximation. In a preferred embodiment, the length of the shank 2 is about 2.5 cm to about 7.0 cm. In a more preferred embodiment, the length of the shank 2 is about 3.0 cm to about 5.0 cm. The width of the shank 2 can also be designed for a specific application. However, in a preferred embodiment, the width of the shank 2 is about 1.5 mm to about 6.0 mm. In a more preferred embodiment, the width of the shank is about 3.0 mm to about 5.0 mm. The cross-sectional shape of the shank 2 can be a square, rectangle, oval, circle, rhombus, or any of a variety of other shapes, or any combination thereof, some examples of which are shown in FIGS. 1C and 1D.

The ratchet teeth 4 can be present on one or more sides and/or can be present on one or more sections along the various sides of the shank. For example, in one embodiment, shown for example in FIG. 1D, the shank can have one or more ratchet teeth 4 on more than one side to secure the device after tissue approximation, while the distal end 14 can have only a few ratchet teeth 4 or have them only on one or a few sides to assist in initiating a tissue approximation. In one method of use of the subject invention, to be discussed below, once a device 50 is assembled and tissue approximation is accomplished, the extraneous distal end 14 of the shank 2 will be removed.

In one embodiment, the shank 2 of the device 50 is generally flattened, as shown in FIGS. 1A, 1B, 1E, and 1F, such that the width 3 of the shank is greater than its thickness 5. In a further embodiment, the shank 2 comprises ratchet teeth 4 on at least one side and along most of the length of the shank 2. In a still further embodiment, there are a multitude of ratchet teeth 4 along the one or more sides of the shank 2 sufficient to provide a surgeon with precise control of the amount of pressure to be applied to the approximated and surrounding tissues.

In a further embodiment, shown for example in FIG. 3A, the shank 2 can have a generally round and/or oval circumferential shape and comprise a plurality of fins 6 along some portion of the shank 2. The fins 6 act similarly to the ratchet teeth 4 discussed above, in that they secure the device after tissue approximation. In this embodiment, the fins 6 can surround all or a part of the circumference of the shank. An example of this embodiment is shown in FIG. 3B, which illustrates a cross-section of FIG. 3A taken along line A-A′.

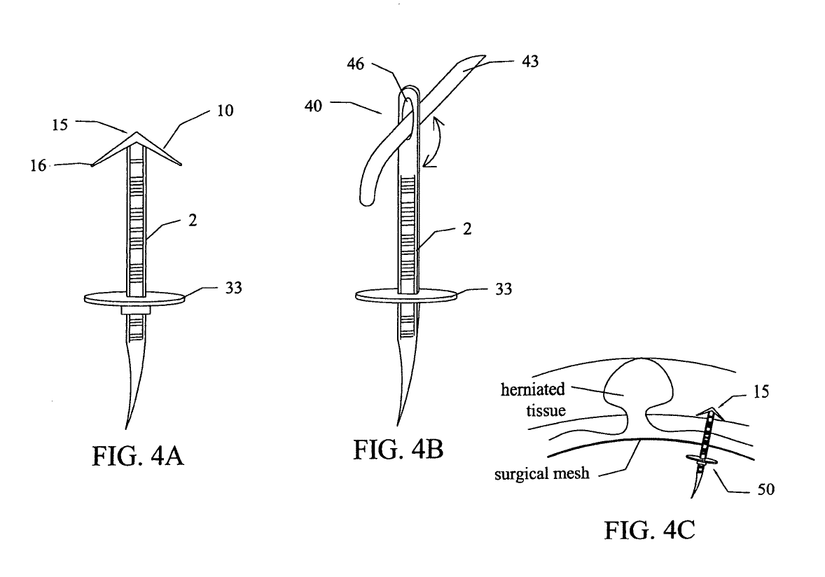

A still further embodiment, shown for example in FIG. 4A, utilizes a shank 2 having a generally round and/or oval circumferential shape and a plurality of round projections or beads 7 along the length of the shank. The beads 7 act similarly to the ratchet teeth 4 and/or fins 6 discussed above, in that they aid in securing the device after tissue approximation. In this embodiment, each bead surrounds the circumference of the shank. An example of this embodiment is shown FIG. 4B, which illustrates a cross-section of FIG. 4A taken along line B-B′.

Utilizing a shank that is substantially round or oval circumference can be advantageous in that it does not require alignment or orientation of the shank relative to a locking button, discussed in more detail below. By further having a plurality of circular or oval fins 6 or beads 7 arranged thereon further negates the need to align or orient the shank 2 prior to engagement with an appropriate locking button. A wide variety of other fin 6 or bead 7 styles and/or configurations can be useful for engaging with an appropriate locking button and securing the device after tissue approximation and will be apparent to those skilled in the art from the description herein. Such modifications and variations are contemplated to be within the scope of the subject invention.

As mentioned previously, after the tissues to be approximated have been drawn or pressed together and a device of the subject invention is secured, the excess portion of the shank 2 can be removed. This can be performed by a variety of techniques. For example, the shank 2 can be cut with scissors, a scalpel or other sharp implement. In one embodiment, the length of the shank 2 of the devices of the subject invention comprise one or more break points 5 or weakened areas where excess shank length can be removed by bending, twisting, etc. along or at a break point 5. In a further embodiment, these break points 5 can have a specific angle, required break force or special procedure for separating the shank 2 to prevent accidental separation of the shank 2 during or after the tissue approximation procedures.

With reference to the Figures, it can further be seen that the proximal end 12 of the shank 2 of these devices can have fixedly attached thereto an anchor button 8 having a front face 9 and a back face 10. The shank 2 can extend from anywhere on the front face 9 of the anchor button 8. For example, in certain embodiments, one or more shanks 2 can be affixed to the front face 9 of the anchor button 8 and radiate from the center of the anchor button 8 or, alternatively, they can be affixed so as to radiate in a preferred pattern from around the circumference of the anchor button 8. A specifically exemplified embodiment utilizes a single shank 2 affixed to the front face 9 of the anchor button 8, offset from the center, possibly along an edge, for example as shown in FIG. 1B. In a still further alternative embodiment, the shank 2 can be made thicker at the junction between the front face 9 and the proximal end 12 of the shank 2 to reduce bending between these components.

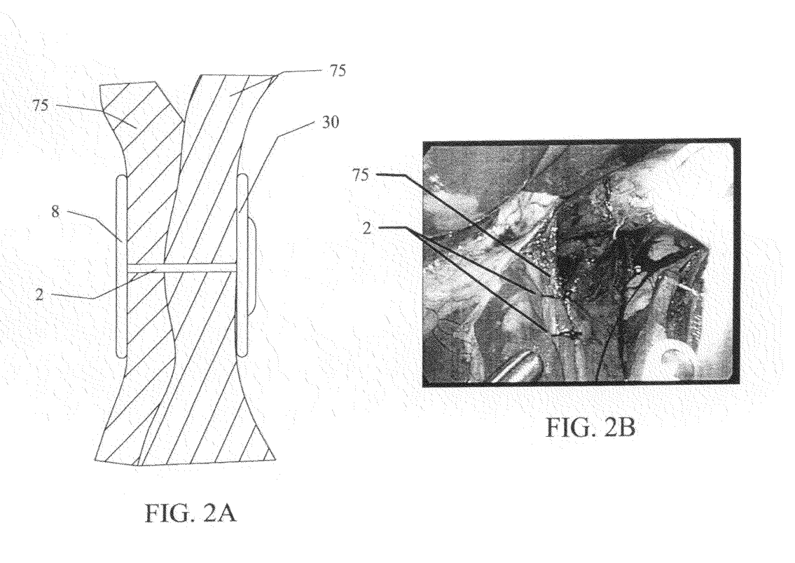

In one embodiment of the device, a single shank 2 is affixed at or near the center of the front face 9 of the anchor button 8, as shown in FIG. 1A. In this embodiment, the shank 2 radiates essentially perpendicularly to the front face 9 of the anchor button 8. In a further embodiment, the anchor button 8 is stiff or firm enough to adequately support the tissues 75 that will be pressed against it for tissue approximation, for example, as shown in FIG. 2A. The ability of the devices of the subject invention to support approximated tissues over a larger area can aid in preventing the pinching and stricture of tissue, as shown for example in FIG. 2B, which can contribute to tearing of tissues when standard suturing cuts tissues or constricts blood flow. The diameter of the anchor button 8, thus the area of support, can vary depending upon the procedure and/or type of tissue approximation for which the device is utilized. In one embodiment, the diameter of the anchor button 8 is about 4.0 mm to about 15.0 mm. In another embodiment, the diameter of the anchor button 8 is about 5.0 mm to about 10.0 mm. In another embodiment, the anchor button 8 diameter is about 7.0 mm to about 9.0 mm.

In an alternative embodiment, the front face 9 of an anchor button 8 can comprise any of a variety of one or more hooks, knobs, teeth, pressure devices, gripping elements, clamps, adhesives, or other stabilizing devices or fixtures to maintain the placement of the anchor button 8 against tissue, as shown for example in FIG. 3A. In one embodiment, an example of which is shown in FIG. 3B, an anchor button 8 comprises two or more hooks 11 capable of inserting or pressing into tissue to stabilize the position of the tissues and/or the device of the subject invention. In a further embodiment, the hooks 11 can be bent, angled or otherwise pointed in the direction of the shank 2, such that when the front face 9 of the anchor button 8 is pressed against tissues, some portion of the ends of the hooks either penetrate or press into tissue 75 sufficiently to ensure that the position of the anchor button 8 and tissues are generally stable relative to each other. This can be particularly useful in the closure of trocar sites to prevent separation of the tensile tissues of the abdominal wall (the fascia) and prevent or limit hernia formation at the trocar sites.

In order to pass the shank 2 of these devices through tissues 75, tissue support apparatuses, or other structures, e.g., surgical mesh, bioabsorbable material(s), sutures and like materials, there must be one or more openings in the tissues. This can be accomplished in a variety of ways including the use of standard surgical devices to create openings through which the shank can be inserted. However, it is desirable to reduce the amount of equipment and devices necessary for tissue approximation, especially in endoscopic procedures. It is also desirable to make tissue approximation efficient, accurate and easy to perform, particularly in such endoscopic procedures.

Therefore, it can be seen from the Figures, particularly FIG. 1 that the distal end 14 of the shank 2 can further comprise a material or end implement 20 capable of puncturing or cutting through tissues to be approximated. This sharp or pointed end implement 20 can be achieved by any of a variety of techniques or methods known to one with skill in the art. For example, the shank end material can be formed or sharpened into a shape capable of puncturing or cutting through or otherwise forming an opening for the shank 2 to follow through. An alternative embodiment utilizes any of various materials, objects or devices that can be coupled or joined with the end of the shank 2 to puncture, cut or otherwise perforate tissues. For certain procedures, the size of the trocar or other installation equipment that could be used with the devices of the subject invention can dictate the size or shape of the distal end or devices affixed thereto. In addition, as mentioned above, in certain embodiments of the subject invention, when the tissue approximation procedure is complete, such that the tissues or other materials have been threaded onto the shank, and the tissue fastening device 50 of the subject invention is fully assembled, any excess distal end of the shank 2 can be removed, which also removes the means that was used for cutting or puncturing the tissues.

A further alternative embodiment of the device utilizes a thread, suture, cord, or similar apparatus affixed or coupled to the distal end 14 of the shank 2. Or, in a still further embodiment, the entire shank 2 can comprise said thread, cord, or similar object, whereby an anchor button 8 is affixed to the proximal end 12. For example, in this embodiment, a textured suture 6, known to those with skill in the art, can be utilized at the distal end of the shank 2, or could comprise the entire shank. Any of various objects or devices for puncturing or cutting through tissues can be affixed, coupled or otherwise joined to the distal end 14 of the thread, suture, cord or similar object. In this embodiment, a surgeon can determine what preferred implement would be suited for the tissue approximation procedure and affix that implement to the thread, cord, etc. at the distal end 14 of the shank. For example, a preferred style or type of surgical needle could be threaded onto the end of the shank for the tissue approximation procedure. At the end of the procedure, if desired, the surgical needle can be removed prior to or after removal of the excess length of shank 2, as described above. In one embodiment, the end implement 20 comprises a needle 21 is affixed, coupled, or otherwise joined to and/or forming the distal end 14 of the shank 2, for example, as shown in FIGS. 1A, 1B, and 1H. In a further preferred embodiment, the needle 21 is contiguous with the distal end 14 of the shank with the juncture point 22 between them being appropriately angled and/or smoothed to ease the passage of the usually wider shank 2 through the opening created by the narrower needle 21. In one embodiment, a “V-20” type needle is coupled or joined to, fixedly attached to and/or forms the distal end 14 of, the shank.

An alternative embodiment utilizes a modified anchor button 8 capable of penetrating tissues, tissue support apparatuses or other structures, e.g., surgical mesh, bioabsorbable material(s), sutures, and similar materials, as shown for example in FIGS. 4A, 4B, and 4C. In one alternative embodiment, the anchor button and is modified to form a toggle-bolt-like configuration with the shank. In this embodiment, shown for example in FIG. 4A, the anchor button 8 comprises two or more extensions or flanges 16 that when affixed at or near the end of the proximal end 12 of the shank 2 generally resemble a “T”-shaped device. In a further embodiment, all or some portion of the back face 10 of the anchor button 8 can be sharpened and/or pointed to facilitate puncturing of tissue or other structures, e.g., surgical mesh. Alternatively, an opening can be created in tissues or structures by methods known in the art and the toggle-end 15 passed into or through the created opening. The flanges 16 can further comprise one or more materials having an elastic resiliency that will permit the permits them to be temporarily bent or otherwise deformed for easier penetration into or through a tissue or other structure with minimal damage thereto. And, once the toggle-end 15 has been positioned, it can automatically return, or can be made to return, to the substantially original, flared configuration, such that the toggle-end will no longer pass back through the opening. A person with skill in the art would be able to determine one or more materials, preferably biocompatible materials, appropriate for the manufacture of the toggle-end and/or shank attached thereto, which can include, for example, plastics, rubbers, silicones, metals, or bioabsorbable materials.

A still further embodiment can utilize a toggle-end 15 anchor button having flanges 16 comprising temperature or chemically-sensitive shape-memory alloys, such as NiTi (Nickel-Titanium), CuZnAl, and CuAlNi. In this embodiment, the toggle-end 15 and/or flanges 16 thereon can be form a shape and size at one temperature (Martensite phase) amendable to being passed through tissues or structures (e.g., sharpened, pointed, serrated, or combinations thereof), but at a different, usually higher temperature (Austenite phase), will attain a different shape configuration (e.g., expanded, flared, opened, or similarly enlarged) to secure the subject device in or against tissues.

A further alternative embodiment utilizes an anchor button comprising an articulated harpoon 40. This embodiment, shown, for example, in FIG. 4B, utilizes a stylet 43 that is movably affixed to the proximal end 12 of a shank 2. The stylet can be sharpened at one or both ends and can further have one or more sharpened edges at one or both ends. In this embodiment, the stylet 43 can be affixed to shank 2 at any point along its length to form a fulcrum 46 allowing the harpoon 40 to move in an up-and-down or “seesaw” fashion, as indicated in FIG. 4B. A wide variety of devices known in the art and useful for affixing the harpoon 40 to the shank 2 in said articulated configuration, e.g., pins, dowels, joints, and the like, will be apparent to those skilled in the art from the description herein and are contemplated to be within the scope of the subject invention. This embodiment can function similarly to a common “toggle bolt” apparatus. With this embodiment, a secondary structural attachment or apparatus can be affixed through, for example, the abdominal wall and the locking button can be secured to that point of attachment. For example, this embodiment could be used to secure mesh to the abdominal wall for repair of a hernia. In that setting, the stylet end 43 of the shank 2 can be passed through abdominal tendons and brought back through the abdominal tendons, such that the anchor button 8 and the stylet 43 are positioned on the same side of the abdominal tendons. The shank 2 can be passed through the mesh and the locking button 30 engaged with the shank, thus, securing the mesh in place.

After completing a tissue approximation with the shank 2 and associated end implement 20, or other means as described above, and anchor button 8 components of the tissue fastening device, the portion of the shank 2 in contact with the approximates tissues must be clamped or closed so that the tissues are securely pressed and held together on the shank 2 to promote the healing process. Preferably, the closing mechanism is easy to use, assists in pressing the tissues against the anchor button 8 and enables the distribution of tension in the tissues to reduce or eliminate tearing or shredding of the approximated tissues. This can be achieved by any of a number of techniques or devices, which would be apparent to a person with skill in the art. For example, the circumference of the distal end 14 of the shank 2 can be secured with various biocompatible and/or absorbable clamps, staples, etc. in conjunction with any of a variety of biocompatible and/or absorbable meshes, pads, etc. Alternatively, all, or part, of the shank 2 can comprise a bendable material such that the distal end, or a portion thereof, can be bent or turned sideways to press against the tissues that are approximated on the proximal end of the shank 2.

In one embodiment, the devices of the subject invention further utilize one or more locking buttons 30, an example of which is shown in FIGS. 1A, 1B, 1E, and 1G. In this embodiment, a locking button 30 has a shank slot 31 through which the shank 2 passes, a contact face 32 that presses the approximated tissues against the front face 9 of the anchor button 8, and a pressure face 33 which can be pushed to move the locking button 30 towards the proximal end 12 of the shank 2 to increase the pressure applied to the approximated tissues between the front face 9 and the contact face 32.

In a still further embodiment, the locking button 30 comprises a mechanism within the shank slot 31 that engages with the shank 2 to hold the locking button 30 in the desired position on the shank 2 to maintain adequate desired pressure on the tissues. The shank slot 31 can be flush with the contact face 32 and/or the pressure face 33 of the locking button, as shown for example, in FIG. 1A. Alternatively, the shank slot 31 may extend outward from the contact face 32 and/or the pressure face 33 of the locking button, as shown for example, in FIG. 1B. It would be apparent to a person with skill in the art that numerous appropriate mechanism(s) can be used with the various embodiments of the shank 2 utilized with the subject invention. For example, various pressure devices, gripping elements, clamps, adhesives, etc. can be utilized with the subject invention to prevent movement of the locking button 30 once positioned on the shank 2. Preferably, these devices hold the locking button 30 securely to prevent accidental disengagement with the shank 2, which could necessitate further surgery to correct the problem.

In one embodiment, the locking mechanism comprises a pawl 35 positioned within the shank slot 31. In yet a further embodiment, the pawl 35 is cantilevered to engage with the ratchet teeth 4 on the shank 2, which advantageously allows the locking button 30 to be advanced towards the proximal end 12 of the shank, but prevents movement backwards towards the distal end 14. Thus, in one method of use, the shank 2 is passed through the shank slot 31 of the locking button 30 where at least one cantilevered pawl 35 engages with at least one of the ratchet teeth 4 on the shank 2 allowing the locking button 30 to be advanced towards the anchor button 8. In this embodiment, the pawl 35 prevents the locking button 30 from being reversed, or moved towards the distal end 14 of the shank 2. In the event that the locking button 30 is advanced too far along the length of the shank 2, the shank 2 can be severed, cut, broken, etc. so that the device can be removed, which could necessitate re-approximation of tissues if too far advanced.

The ratchet teeth 4 can comprise any of a variety of configurations suitable for locking the shank 2 at a desired length and compatible with a pawl 35. It can be advantageous to utilize cantilevered ratchet teeth 4 with a pawl so that the shank length can be securely fixed. However, in other situations, it can be more beneficial to have ratchet teeth 4 and a compatible pawl 35 that afford some adjustability to the length of the shank 2. Thus, in an alternative embodiment, one or more of the plurality of ratchet teeth are modified so as to secure the shank 2 with the pawl 35 when the pawl is moved towards the proximal end 12 of the shank; but, can also permit the shank to be pulled through the pawl towards the distal end 14, if adjustment of the shank length is necessary. To accomplish this duality of adjustability, the ratchet teeth can be cantilevered, but at an angle that permits coupling of the ratchet teeth when the pawl is moved towards the proximal end, but can also permit the pawl and/or teeth to be slightly bent or deformed with sufficient applied force, permitting them to be uncoupled and realigned when the pawl is pulled towards the distal end 14. In this embodiment, the ratchet teeth and/or the pawl can comprise one or more materials having, or be designed in such a way as to have, some elasticity, resiliency, or bendability to permit the pawl and ratchet teeth to cross over each other, in either direction, without damage to either.

A still further embodiment of the locking button 30 can utilize a release mechanism with the pawl that permits it to be disengaged from the shank and the respective locking mechanisms thereon, for more refined positioning. For example, the pawl can be affixed within the shank slot such that it can be pivoted or pushed in a direction away from the shank. Such devices are known in the art and a person having skill in the art and benefit of the subject disclosure would be able to devise any of a variety of pawl release mechanisms. Such obvious variations are considered to be within the scope of the subject invention.

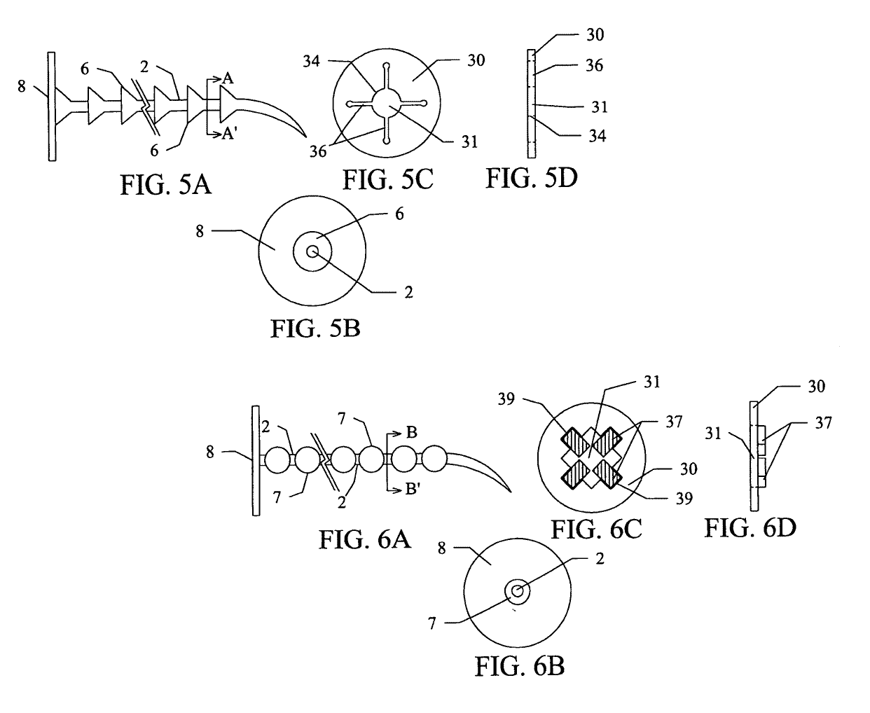

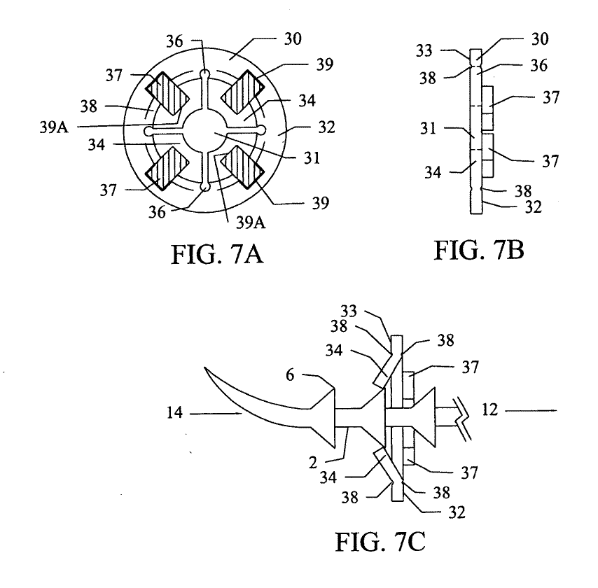

An alternative embodiment of a locking button 30, shown for example in FIG. 5C, can be utilized with any of shanks presented herein, but can be particularly useful with shanks having fins 6 or beads 7, or similar protrusions, as presented above. This embodiment comprises a lock button 30 being generally flat on the contact face 32 and the pressure face 33, wherein the periphery of the shank slot 31 forms one or more shank slot tabs 34 that can act as one or more pawls for engagement with fins 6, beads 7, or similar structures. In this embodiment, the locking button 30 or, at least some portion thereof around the shank slot periphery, can comprise a semi-rigid or semi-elastic material. Further embodiments include one or more notches 36, as shown, for example, in FIGS. 5C, 5D, and 7A, that can permit the shank slot tabs 34 to be pushed or moved towards the pressure face 33 side of the locking button, as will be explained below.

A still further embodiment makes use of one or more shoulder flanges 37 located around the periphery of, and extending at least partially into, the shank slot 31, for example, as shown in FIGS. 6C and 6D. In this embodiment, a shank and its associated fins, beads or similar structures can be pushed and/or pulled through the shank slot. As shown in FIG. 7C, the fins, beads, or similar structures thereon or around the shank, when pushed against the shank slot, cause at least a portion of the one or more shank slot tabs 34, or the shoulder flanges 37 in the alternative embodiment, to bend, temporarily deform, or otherwise give-way to allow passage of the fins, beads, or similar structures. In an alternative embodiment, the fins 6, beads 7, or similar structures can also comprise a semi-rigid or semi-elastic material that can also be bent, or temporarily deformed to permit their passage past the shank slot tabs 34. In this way, the shank 2 can be pushed through the shank slot 31, forcing the fins 6, beads 7 or similar structures past or through the one or more shank slot tabs 34 until the desired distance is achieved between the anchor button 8 and the locking button 30 to complete a tissue approximation.

In yet a further embodiment, a locking button 30 can comprise a combination of one or more moveable shank slot tab 34 and one or more shoulder flanges 37, discussed above. In this embodiment, shown for example in FIGS. 7A, 7B and 7C, the shank slot tabs can be affixed to a locking button 30 by a flexible hinge 38 that permits the one or more shank slot tabs 34 to be moved or swung out of position when contacted by a shank 2 having one or more teeth, beads, fins, or similar structures. The hinge 38 can comprise any of a variety of apparatuses known in the art. For example, FIGS. 7A, 7B, and 7C illustrate an embodiment where the shank slot tabs are affixed to the locking button by one or more pre-formed areas that are thinner or of less thickness than other areas of the locking button. When using certain materials, known to those with skill in the art, these one or more thinner areas can act as a hinge 38 to provide a measure of flexibility, permitting the shank slot tabs 34 to be moved or pushed out of the position shown in FIG. 7B and in the direction of the pressure face 33, as shown in FIG. 7C. This movement of the shank slot tabs 34 increases the diameter of the shank slot 31 permitting passage of the shank and any structures thereon, as seen for example in FIG. 7C. However, in order to secure the shank within the shank slot 31 and prevent movement of the shank in the direction of the contact face 32, a further embodiment utilizes one or more shoulder flanges 37 affixed around the periphery of the shank slot 31 on the contact face 32. In this embodiment, shown for example in FIG. 7A, the one or more shoulder flanges 37 are positioned so that they traverse across at least a portion of the hinge 38 on the contact face side 32 of the shank slot tabs 34. This affixed section 39 secures the shoulder flange 37 to the locking button 30. The unaffixed section 39A that extends along the contact face 32 side of the shank slot tabs 34 assumes the role of a stop guard or block to reduce or prevent movement of the shank slot tabs 34 in the direction of the contact face 32. In this embodiment, the distal end 14 of a shank 2 can be introduced into the shank slot 31 from the contact face side 32. As the locking button 30 progresses towards the proximal end 12 of the shank, any teeth, fins, beads, or other structures on the shank cause the shank slot tabs 34 to be moved or pushed, with the aid of the one or more hinges 38, towards the distal end 14 of the shank, as shown for example in FIG. 7C. Once the shank 2 is properly positioned within the shank slot (i.e., tissues and/or other structures are approximated), the shank can be adjusted either distally 14 or proximally 12, if necessary, to disengage the shank slot tabs from structures on the shank. This permits the shank slot tabs 34 to resume their prior position, shown for example in FIG. 7B, that being generally flush with the contact face 32, which causes the shank slot to resume its prior smaller diameter. Attempting to push and/or pull or otherwise move the shank in the proximal direction 12 will cause the fins, teeth, beads, or other structures on the shank to also move the shank slot tabs in the proximal direction causing them to be pressed against the shoulder flanges 37, which block the movement of the shank slot tabs. This ensures that the shank slot 31 maintains a diameter that prevents passage of any teeth, fins, beads, or other structures on the shank. Thus, the shank 2 becomes locked against movement in the proximal direction.

The diameter of the locking button 30 can vary depending upon the procedure and type of tissue approximation for which the device is utilized. In addition, the diameter of the locking button 30 and the diameter of the anchor button 30, discussed above, can be different. For example, in certain procedures, it can be advantageous for the diameter of the locking button 30 to be larger or smaller than the diameter of the anchor button 30. In one embodiment, the diameter of the locking button 30 is about 4.0 mm to about 15.0 mm. In another embodiment, the diameter of the locking button 30 is about 5.0 mm to about 10.0 mm. In another embodiment, the locking button 30 diameter is about 7.0 mm to about 9.0 mm.

As with the anchor button 8, the contact face 32 of the locking button 30 can also comprise any of a variety of one or more hooks, knobs, teeth, pressure devices, gripping elements, clamps, adhesives, or other stabilizing devices or fixtures to aid in maintaining the position of tissues and/or the placement of the locking button 30 against tissue. In one embodiment, shown for example in FIG. 3A, the contact face 32 of a locking button 30 can comprise one or more teeth 13 capable of inserting or pressing into tissue to stabilize the position of the tissues and/or the device of the subject invention.

The devices and procedures of the subject invention are useful in a variety of surgical procedures. These devices and procedures can be particularly useful with tissues that are subjected to pulling or stretching stresses. For example, the devices and methods are useful during laparoscopic repair of hernias, for example hiatal hernias. The device can distribute tissue stresses over a greater area, which can reduce or eliminate the failure of tissue approximations, often caused by tissues being pulled or lacerated from standard surgical sutures. In addition, the advantageous elimination of intracorporeal knot-tying during tissue approximation allows the device to be particularly useful in endoscopic surgeries.

All patents, patent applications, provisional applications, and publications referred to or cited herein are incorporated by reference in their entirety, including all figures and tables, to the extent they are not inconsistent with the explicit teachings of this specification.

It should be understood that the examples and embodiments described herein are for illustrative purposes only and that various modifications or changes in light thereof will be suggested to persons skilled in the art and are to be included within the spirit and purview of this application.

1. A biocompatible fastening device comprising:
an anchor button;
an elongated shank having a proximal end fixedly attached to the anchor button and a distal end joined to an end implement capable of penetrating materials to be fastened to secure them onto the shank;
a locking button having a shank slot therethrough; and
a locking mechanism coupled with said shank slot and capable of engaging with and securing the position of the shank after the distal end is passed through the shank slot,
such that materials penetrated by the end implement and secured on the shank can be compressed between the anchor button and the locking button when the shank is pulled through the shank slot.

2. The fastening device according to claim 1, wherein the end implement comprises a needle.

3. The fastening device, according to claim 1, wherein said shank further comprises a plurality of ratchet teeth.

4. The fastening device, according to claim 1, wherein the locking mechanism within the shank slot comprises a cantilevered pawl.

5. The fastening device, according to claim 1, wherein the shank further comprises a plurality of fins.

6. The fastening device, according to claim 1, wherein the shank further comprises a plurality of beads.

7. The fastening device, according to claim 1, wherein the locking mechanism comprises a semi-rigid shank slot periphery.

8. The fastening device, according to claim 1, wherein the locking mechanism comprises one or more shoulder flanges.

9. The fastening device, according to claim 1, wherein the locking mechanism comprises one or more shank slot tabs in conjunction with one or more shoulder flanges

10. The fastening device, according to claim 9, wherein the one or more shank slot tabs are affixed to the locking button via one or more hinges.

11. The fastening device, according to claim 10, wherein the one or more hinges comprise one or more pre-formed areas of the locking button having a thickness that permits bending of the material at the pre-formed area.

12. The fastening device, according to claim 1, comprising one or more bio-absorbable materials.

13. The fastening device, according to claim 1, wherein the length of the shank is between about 2.5 cm to about 7.0 cm.

14. The fastening device, according to claim 1, wherein the length of the shank is between about 3.0 cm to about 5.0 cm.

15. The fastening device, according to claim 1, wherein the width of the shank is between about 1.5 mm to about 6 mm.

16. The fastening device, according to claim 1, wherein the width of the shank is between about 3.0 mm to about 5.0 mm.

17. The fastening device, according to claim 1, further comprising one or more break points along the length of the shank.

18. The fastening device, according to claim 1, wherein the diameter of the anchor button is about 4.0 mm to about 15.0 mm.

19. The fastening device, according to claim 1, wherein the diameter of the anchor button is about 5.0 mm to about 10.0 mm.

20. The fastening device, according to claim 1, wherein the diameter of the anchor button is about 7.0 mm to about 9.0 mm.

21. The fastening device, according to claim 1, wherein the diameter of the locking button is about 4.0 mm to about 15.0 mm.

22. The fastening device, according to claim 1, wherein the diameter of the locking button is about 5.0 mm to about 10.0 mm.

23. The fastening device, according to claim 1, wherein the diameter of the locking button is about 7.0 mm to about 9.0 mm.

24. The fastening device, according to claim 1, wherein the anchor button further comprises one or more stabilizing devices.

25. The fastening device, according to claim 24, wherein the stabilizing device comprises one or more hooks, knobs, teeth, clamps, or adhesives.

26. The fastening device, according to claim 1, wherein the locking button further comprises one or more stabilizing devices.

27. The fastening device, according to claim 26, wherein the stabilizing device comprises one or more hooks, knobs, teeth, clamps, or adhesives.

28. The fastening device, according to claim 1, wherein the anchor button comprises a toggle-end.

29. The fastening device, according to claim 28, wherein the backface of the toggle-end comprises one or more sharpened edges and/or points.

30. The fastening device, according to claim 1, wherein the anchor button comprises an articulated harpoon.

31. A method for approximating tissues utilizing a biocompatible tissue approximating device comprising:
an anchor button;
an elongated shank having a proximal end fixedly attached to the anchor button and a distal end comprising an end implement capable of penetrating materials to be fastened to secure them onto the shank;
a locking button having a shank slot therethrough; and
a locking mechanism secured within said shank slot and capable of engaging with and securing the position of the shank after the distal end is passed through the shank slot,
wherein said method comprises penetrating one or more tissues with the end implement on the distal end of the shank to permit the length of the shank to pass through the tissues; passing the distal end of the shank through the shank slot until the shank engages with the locking mechanism therein; and moving the locking button towards anchor button, thereby compressing the tissues, which have been approximated on the shank, between the locking button and the anchor button.

32. The method, according to claim 31, wherein said method further comprises approximating one or more surgical apparatuses.

33. The method, according to claim 22, wherein said surgical apparatuses comprise surgical mesh or other tissue support structures.

34. The method, according to claim 31, further comprising the removal of excess shank length after tissues have been sufficiently compressed between the anchor button and the locking button.

35. A kit comprising:
at least one biocompatible tissue approximating device comprising
an anchor button;
an elongated shank having a proximal and a distal end, wherein the proximal end is fixedly attached to the anchor button;
a locking button having a shank slot therethrough;
a locking mechanism secured within said shank slot and capable of engaging with and securing the position of the shank after the distal end is passed through the shank slot, and
at least one needle for affixing to the distal end of the shank.
